(19) United States
(12) Patent Application Publication
(10) Pub. No.: US 2019/0053713 A1
(43) Pub. Date: Feb. 21, 2019

(54) Device with Skin Temperature Sensor and Corresponding Methods

(71) Applicant: Motorola Mobility LLC, Chicago, IL (US)

(72) Inventors: Scott DeBates, Crystal Lake, IL (US); Doug Lautner, Round Lake, IL (US); Navin Dabhi, Sunnyvale, CA (US)

(21) Appl. No.: 15/681,226

(22) Filed: Aug. 18, 2017

(51) Int. Cl. A61B 5/01; G01K 1/02; A61B 5/00; F24F 13/30

(52) U.S. Cl. CPC A61B 5/01; F24F 13/30; A61B 5/0008; G01K 1/024

(57) ABSTRACT

A device includes a skin temperature sensor, one or more processors, and a wireless communication circuit. The one or more processors determine, with signals received from the skin temperature sensor, whether a skin temperature that is outside a predefined skin temperature range. Where the skin temperature is outside the predefined skin temperature range, the one or more processors one or both of cause the wireless communication circuit to transmit a climate adjustment request to a climate control device and actuate a local cooling device.

### BACKGROUND

### Technical Field

This disclosure relates generally to devices, and more particularly to wireless communication devices.

### Background Art

Electronic devices, and in particular portable, wireless communication devices, are becoming increasingly technologically advanced. In response, people are becoming more dependent upon their portable electronic devices. Only a few years ago a mobile telephone was a novelty item used only for making telephone calls. By contrast, people today rely upon “smartphones” to keep up with their calendars, address books, music collections, photo collections, and so forth. Modern smartphones have evolved to the point that they serve as a computing device, entertainment device, productivity device, and communication device, all while neatly slipping into a pocket.

These smaller, yet more powerful, devices are being used for many different applications in many different environments. It would be advantageous to have new applications for electronic devices to detect certain environments offer enhanced performance within a given environment.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present disclosure.

FIG. 1 illustrates one explanatory electronic device in accordance with one or more embodiments of the disclosure.

FIG. 2 illustrates one or more explanatory sensors, which can be used in various sets, subsets, and combinations, in one or more explanatory devices configured in accordance with one or more embodiments of the disclosure.

FIG. 3 illustrates one explanatory cooling system suitable for use in a device in accordance with one or more embodiments of the disclosure.

FIG. 4 illustrates another explanatory cooling system suitable for use in a device in accordance with one or more embodiments of the disclosure.

FIG. 5 illustrates an explanatory system and one or more method steps in accordance with one or more embodiments of the disclosure.

FIG. 6 illustrates one explanatory method in accordance with one or more embodiments of the disclosure.

FIG. 7 illustrates another explanatory electronic device in accordance with one or more embodiments of the disclosure.

FIG. 8 illustrates another explanatory system and one or more method steps in accordance with one or more embodiments of the disclosure.

FIG. 9 illustrates an explanatory electronic device attachment in accordance with one or more embodiments of the disclosure.

FIG. 10 illustrates one explanatory modular system, with an explanatory attachment detached from an electronic device, in accordance with one or more embodiments of the disclosure.

FIG. 11 illustrates the explanatory modular system of FIG. 10, but with the attachment coupled to the electronic device in accordance with one or more embodiments of the disclosure.

FIG. 12 illustrates another explanatory modular system, with an explanatory attachment detached from an electronic device, in accordance with one or more embodiments of the disclosure.

FIG. 13 illustrates the explanatory modular system of FIG. 12, but with the attachment coupled to the electronic device in accordance with one or more embodiments of the disclosure.

FIG. 14 illustrates yet another explanatory system and one or more method steps in accordance with one or more embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DRAWINGS

Before describing in detail embodiments that are in accordance with the present disclosure, it should be observed that the embodiments reside primarily in combinations of method steps and apparatus components related to sensing a skin temperature and then one or both of sending a climate adjustment signal to a remote device and/or actuation of a local cooling system. Any process descriptions or blocks in flow charts should be understood as representing modules, segments, or portions of code that include one or more executable instructions for implementing specific logical functions or steps in the process. Alternate implementations are included, and it will be clear that functions may be executed out of order from that shown or discussed, including substantially concurrently or in reverse order, depending on the functionality involved. Accordingly, the apparatus components and method steps have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

Embodiments of the disclosure do not recite the implementation of any commonplace business method aimed at processing business information, nor do they apply a known business process to the particular technological environment of the Internet. Moreover, embodiments of the disclosure do not create or alter contractual relations using generic computer functions and conventional network operations. Quite to the contrary, embodiments of the disclosure employ methods that, when applied to electronic device and/or user interface technology, improve the functioning of the electronic device itself by and improving the overall user experience to overcome problems specifically arising in the realm of the technology associated with electronic device user interaction.

It will be appreciated that embodiments of the disclosure described herein may be comprised of one or more conventional processors and unique stored program instructions that control the one or more processors to implement, in conjunction with certain non-processor circuits, some, most, or all of the functions of detecting skin temperature, actuation of a local cooling system, or delivering messages to companion devices as described herein. The non-processor circuits may include, but are not limited to, a radio receiver, a radio transmitter, signal drivers, clock circuits, power source circuits, and user input devices. As such, these functions may be interpreted as steps of a method to perform controlling local or remote heating or cooling equipment in response to detected skin temperatures. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used. Thus, methods and means for these functions have been described herein. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ASICs with minimal experimentation.

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions.

As used herein, components may be “operatively coupled” when information can be sent between such components, even though there may be one or more intermediate or intervening components between, or along the connection path. The terms “substantially” and “about” are used to refer to dimensions, orientations, or alignments inclusive of manufacturing tolerances. Thus, a “substantially orthogonal” angle with a manufacturing tolerance of plus or minus two degrees would include all angles between 88 and 92, inclusive. Also, reference designators shown herein in parenthesis indicate components shown in a figure other than the one in discussion. For example, talking about a device (10) while discussing figure A would refer to an element, 10, shown in figure other than figure A.

Embodiments of the disclosure contemplate that the human body functions most efficiently within a fairly narrow temperature range having a mean of about 98.6 degrees Fahrenheit. Embodiments of the disclosure also contemplate that the human body's internal temperature sensor and temperature regulation mechanisms, for the most part, do a fairly good job of maintaining a constant body temperature. Illustrating by example, during exercise a person's breathing rate increases when the body calls for more oxygen. The body also produces sweat, which in proper conditions evaporates from skin, thereby removing heat.

However, there are circumstances in which the body's internal mechanisms are less than effective. For instance, when a person sweats too excessively, there may be an insufficient amount of water remaining in the body to sufficiently cool the person. Similarly, in very humid environments the sweat may not evaporate sufficiently to remove the necessary heat to cool the skin. In cooler climates, the body may use its limited energy reserves to warm internal organs rather than the skin. In either condition, it is possible for a person's skin temperature to rise above, or fall below, a predefined skin temperature range that is optimal for health. When excessively high—or excessively low—skin temperature occurs, this can have adverse effects of human bodily function.

Embodiments of the disclosure work to resolve this problem by providing a device that includes a skin temperature sensor. As will be shown below, the device can be configured as a headband, garment, electronic device attachment, or in other form factors. One or more processors are operable with the skin temperature sensor. When the one or more processors receive signals from the skin temperature sensor indicating that the skin temperature is outside of a predefined skin temperature range, the one or more processors can take action to reduce—or increase—the skin temperature as necessary.

In one embodiment, the one or more processors take action by causing a wireless communication device to transmit a climate adjustment request to a climate control device. In another embodiment, where the device is equipped with a local cooling system, the one or more processors can actuate the local cooling system when the skin temperature is above a predefined upper skin temperature threshold of the predefined skin temperature range. Of course, combinations of the two can occur as well. Moreover, other actions will be obvious to those of ordinary skill in the art having the benefit of this disclosure. If, for example, the device is equipped with a local warming system, the one or more processors can actuate the local warming system when the skin temperature is below a predefined lower skin temperature threshold of the predefined skin temperature range, and so forth.

Accordingly, in one or more embodiments a device includes a skin temperature sensor to continuously measure a person's skin temperature. In one or more embodiments, the device also includes a wireless communication circuit configured to interact with remote “Internet of Things” devices, such as a heating ventilation and air conditioning (HVAC) system, fan, heater, or other climate control device. In one or more embodiments, the device can be directly attached to the person's skin. In other embodiments, the device can be configured in, or as an attachment to, another electronic device. Other configurations will be obvious to those of ordinary skill in the art having the benefit of this disclosure.

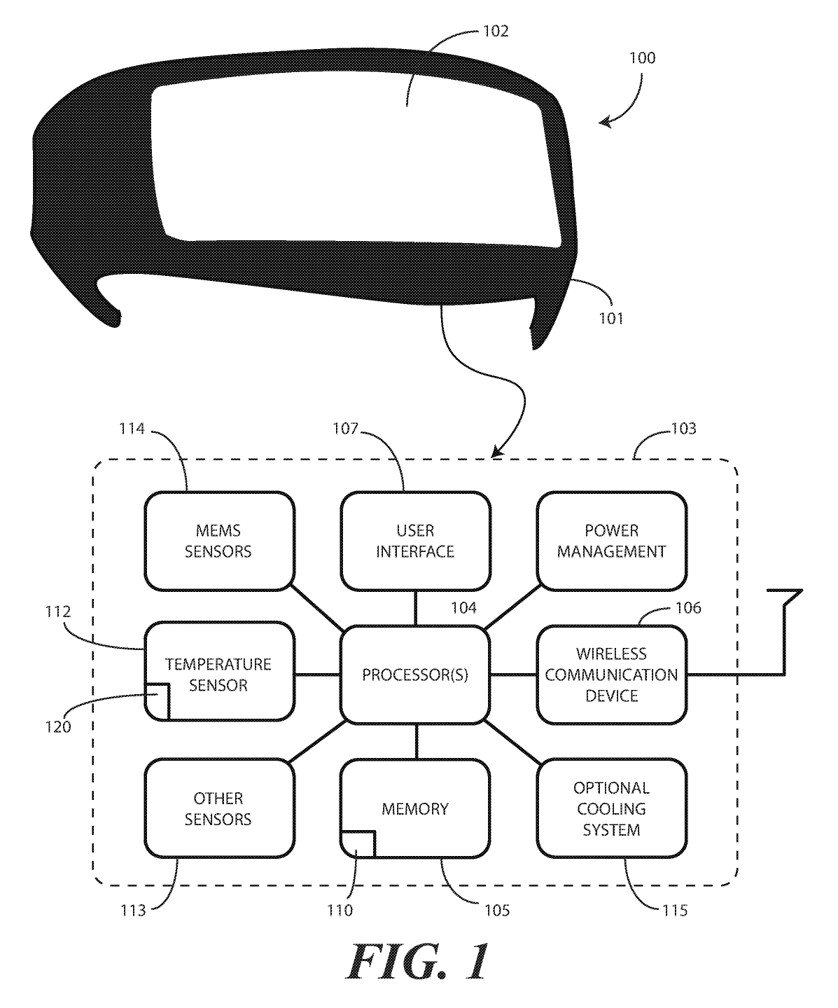

Turning now to FIG. 1, illustrated therein is one explanatory device 100 configured in accordance with one or more embodiments of the disclosure. The device 100 of FIG. 1 is configured as a “wearable” device, meaning that a user can wear the device 100 directly against the skin or clothing. In this illustrative embodiment, the device 100 is configured as a headband. However, it can be configured in other ways as well. Illustrating by example, the device 100 could be configured as a wristband in another embodiment. The device 100 could be configured as a waistband or chest band in yet another embodiment. The device 100 could be configured as an ear bud in still another embodiment. Accordingly, a headband is an example for illustration. Other configurations for the device 100 will be obvious to those of ordinary skill in the art having the benefit of this disclosure.

In one or more embodiments, the device 100 includes a housing 101. The housing 101 can include one or more housing portions, such as a first housing portion and a second housing portion. In this illustrative embodiment, the housing 101 is disposed about the periphery of an optional display 102.

A block diagram schematic 103 of the device 100 is also shown in FIG. 1. In one embodiment, the device 100 includes one or more processors 104. The one or more processors 104 are operable with the display 102 and other components of the device 100. The one or more processors 104 can include a microprocessor, a group of processing components, one or more ASICs, programmable logic, or other type of processing device. The one or more processors 104 can be operable with the various components of the device 100. The one or more processors 104 can be configured to process and execute executable software code to perform the various functions of the device 100.

A storage device, such as memory 105, can optionally store the executable software code used by the one or more processors 104 during operation. The memory 105 may include either or both static and dynamic memory components, may be used for storing both embedded code and user data. The software code can embody program instructions and methods to operate the various functions of the device 100, and also to execute software or firmware applications and modules. The one or more processors 104 can execute this software or firmware, and/or interact with modules, to provide device functionality.

As noted, in one or more embodiments the device 100 includes a display 102, which may optionally be touch-sensitive. In one embodiment where the display 102 is touch-sensitive, the display 102 can serve as a primary user interface 107 of the device 100. In other embodiments, the user interface 107 may comprise buttons, touch sensitive surfaces, or other user interface devices in the absence of a display 102. Users can deliver user input to the user interface 107 by delivering touch input from a finger, stylus, or other objects disposed proximately with the user interface 107 in one or more embodiments. In other embodiments, the user interface 107 may be voice activated. Other configurations of user interfaces will be obvious to those of ordinary skill in the art having the benefit of this disclosure.

Where a display 102 is included, in one embodiment the display 102 is configured as an organic light emitting diode (OLED) display. However, it should be noted that other types of displays would be obvious to those of ordinary skill in the art having the benefit of this disclosure. In one embodiment, the display 102 includes an electroluminescent layer or light-emitting diode (LED) backlighting layer disposed beneath the display 102 to project light through the display 102. The display 102 can adaptively present text, graphics, images, user actuation targets, data, and controls along the display surface.

In this illustrative embodiment, the device 100 also includes a wireless communication circuit 106 that can be configured for wired or wireless communication with one or more other devices or networks. The networks can include a wide area network, a local area network, and/or personal area network. Examples of wide area networks include GSM, CDMA, W-CDMA, CDMA-2000, iDEN, TDMA, 2.5 Generation 3GPP GSM networks, 3rd Generation 3GPP WCDMA networks, 3GPP Long Term Evolution (LTE) networks, and 3GPP2 CDMA communication networks, UMTS networks, E-UTRA networks, GPRS networks, iDEN networks, and other networks.

The wireless communication circuit 106 may also utilize wireless technology for communication, such as, but are not limited to, peer-to-peer or ad hoc communications such as HomeRF, Bluetooth and IEEE 802.11 (a, b, g or n); and other forms of wireless communication such as infrared technology. The wireless communication circuit 106 can include wireless communication circuitry, one of a receiver, a transmitter, or transceiver, and one or more antennas.

The one or more processors 104 can be responsible for performing the primary functions of the device 100. For example, in one embodiment the one or more processors 104 comprise one or more circuits operable with one or more user interface devices, which can include the display 102, to present presentation information to a user. The executable software code used by the one or more processors 104 can be configured as one or more modules 110 that are operable with the one or more processors 104. Such modules 110 can store instructions, control algorithms, and so forth. While these modules 110 are shown as software stored in the memory 105, they can be hardware components or firmware components integrated into the one or more processors 104 as well.

Other components can be included with the device 100. The other components can be operable with the one or more processors 104 and can include input and output components associated with a user interface 107, such as power inputs and outputs, audio inputs and outputs, and/or mechanical inputs and outputs. The other components can include output components such as video, audio, and/or mechanical outputs. For example, the output components may include a video output component or auxiliary devices including a cathode ray tube, liquid crystal display, plasma display, incandescent light, fluorescent light, front or rear projection display, and light emitting diode indicator. Other examples of output components include audio output components such as a loudspeaker disposed behind a speaker port or other alarms and/or buzzers and/or a mechanical output component such as vibrating or motion-based mechanisms. Still other components will be obvious to those of ordinary skill in the art having the benefit of this disclosure.

One or more sensor circuits are operable with the one or more processors 104 in one or more embodiments. These sensor circuits can include one or more skin temperature sensors 112. The one or more skin temperature sensors 112 can detect the amount of thermal energy being generated by the skin of a person using the device 100. Examples of skin temperature sensors 112 include thermistors, thermocouples, thermometers, resistive temperature detectors, silicon band gap temperature sensors, integrated circuit temperature sensors, bimetallic thermostats, infrared signal sensors, and so forth. In one or more embodiments, the skin temperature sensors 112 are disposed along the housing 101 of the device 100 so that they can directly contact the skin of the user.

The one or more skin temperature sensors 112, in one embodiment, also include a skin sensor 120. In one embodiment, a skin sensor 120 is configured to determine when the one or more skin temperature sensors 112 are proximately located with the skin of a user. For example, where the device 100 is configured as a headband and the headband is being worn, in one or more embodiments this can be detected by the skin sensor 120. Moreover, temperature measurements by the one or more skin temperature sensors 112 can be triggered as a function of the skin sensor 120 detecting the device 100 being proximately located or touching the skin of the user. Still other examples of thermal energy sensors will be obvious to those of ordinary skill in the art having the benefit of this disclosure.

One or more other sensors 113 can be included with the device 100. The one or more other sensors 113 can also be configured to sense or determine physical parameters indicative of conditions in an environment about the device 100. Illustrating by example, the one or more other sensors 113 can include devices for determining information such as motion, bearing, location, acceleration, orientation, proximity to people and other objects, incident light amounts, and so forth. The one or more other sensors 113 can include various combinations of microphones, location detectors, motion sensors, physical parameter sensors, temperature sensors, barometers, proximity sensor components, proximity detector components, wellness sensors, touch sensors, cameras, audio capture devices, and so forth.

The one or more other sensors 113 can also include a touch pad sensor, a touch screen sensor, a capacitive touch sensor, and one or more switches. The one or more other sensors 113 can also include audio sensors and video sensors (such as a camera). The one or more other sensors 113 can also include motion detectors, such as one or more accelerometers or gyroscopes. The motion detectors can detect movement, and direction of movement, of the device 100 by a user. The one or more other sensors 113 can also be used to detect gestures. For example, the other one or more other sensors 113 can include one or more proximity sensors that detect the gesture of a user waving a hand above the display 102. In yet another embodiment, the accelerometer can detect gesture input from a user lifting, shaking, or otherwise deliberately moving the device 100. It should be clear to those of ordinary skill in the art having the benefit of this disclosure that additional sensors can be included as well. Some of these components can be configured as Micro-Electro-Mechanical System (MEMS) sensors 114. Moreover, other types of sensors 113 will be obvious to those of ordinary skill in the art having the benefit of this disclosure.

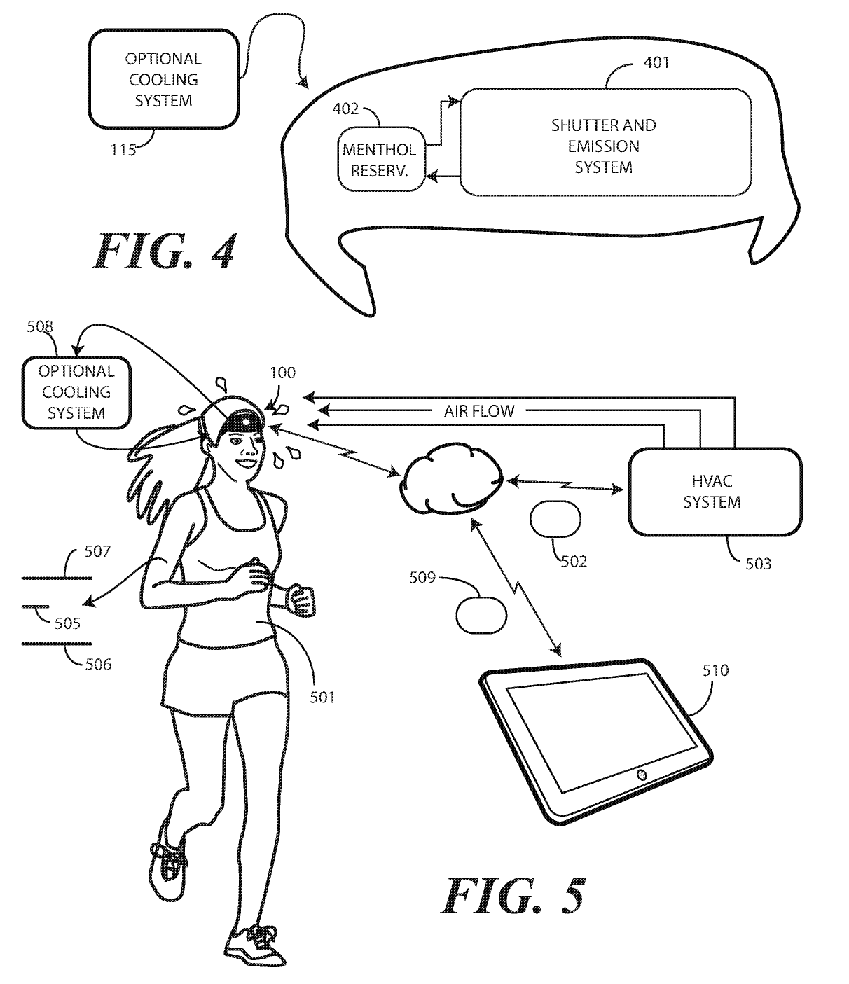

In one or more embodiments, a local cooling system 115 can be included with the device 100. Examples of local cooling systems 115 will be described in more detail below with reference to FIGS. 3 and 4. In one or more embodiments, the one or more processors 104 actuate the local cooling system 115 when the skin temperature is above a predefined upper skin temperature threshold, as will be described in more detail below. In one or more embodiments, the local cooling system 115 comprises a sheet mixture of polymer, menthol and preservatives to keep them intact. Menthol has ability to trigger the cold-sensitive receptors in the skin is responsible for cooling sensation thereby reducing body temperature. The local cooling system 115 can also include a processor to accept commands such as activate or deactivate.

It is to be understood that FIG. 1 is provided for illustrative purposes only and for illustrating components of one explanatory device 100 in accordance with embodiments of the disclosure, and is not intended to be a complete schematic diagram of the various components required for an electronic device. Therefore, other electronic devices in accordance with embodiments of the disclosure may include various other components not shown in FIG. 1, or may include a combination of two or more components or a division of a particular component into two or more separate components, and still be within the scope of the present disclosure.

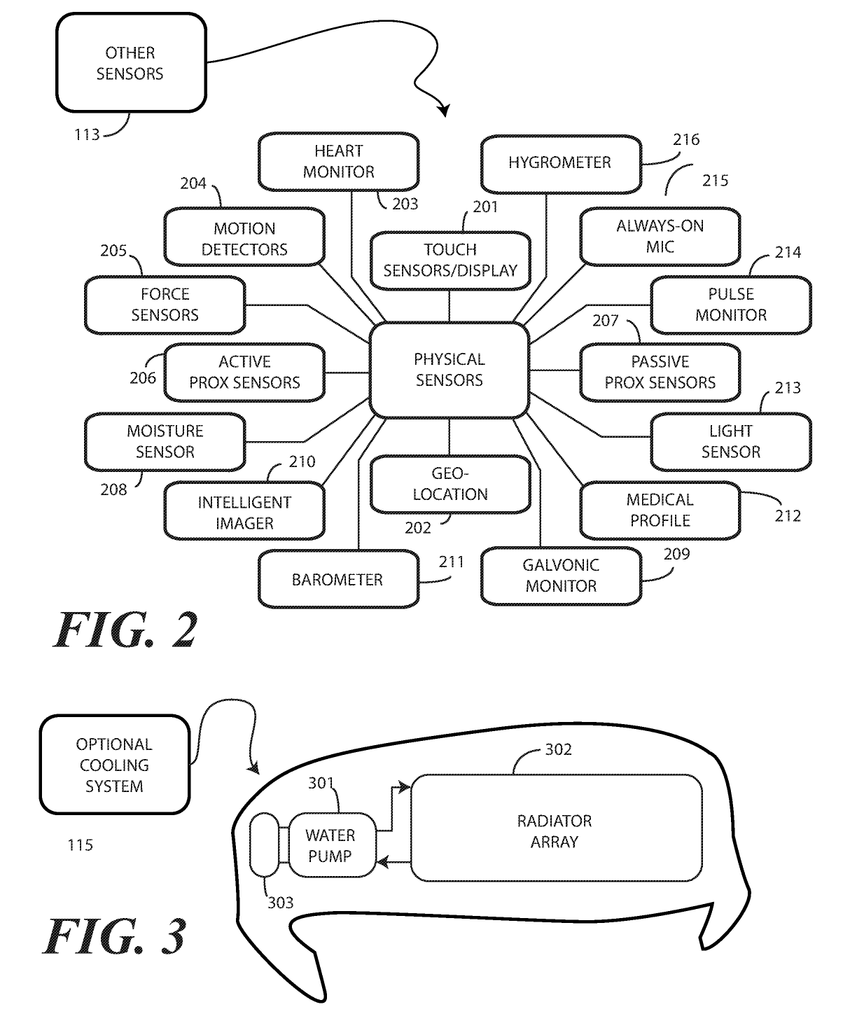

Turning now to FIG. 2, illustrated therein are various examples of the one or more other sensors 113 described above with reference to FIG. 1. As noted above, one or more other sensors 113 to sense or determine physical parameters indicative of conditions in an environment about an electronic device. FIG. 2 illustrates several examples one or more other sensors 113. It should be noted that those shown in FIG. 2 are not comprehensive, as others will be obvious to those of ordinary skill in the art having the benefit of this disclosure. Additionally, it should be noted that the various one or more other sensors 113 shown in FIG. 2 could be used alone or in combination. Accordingly, many electronic devices will employ only subsets of the one or more other sensors 113 shown in FIG. 2, with the particular subset defined by device application.

A first example of a physical sensor is a touch sensor 201. The touch sensor 201 can include a capacitive touch sensor, an infrared touch sensor, resistive touch sensors, or another touch-sensitive technology. Capacitive touch-sensitive devices include a plurality of capacitive sensors, e.g., electrodes, which are disposed along a substrate. Each capacitive sensor is configured, in conjunction with associated control circuitry, e.g., the one or more processors (104), to detect an object in close proximity with—or touching—the surface of the display or the housing of an electronic device by establishing electric field lines between pairs of capacitive sensors and then detecting perturbations of those field lines.

The electric field lines can be established in accordance with a periodic waveform, such as a square wave, sine wave, triangle wave, or other periodic waveform that is emitted by one sensor and detected by another. The capacitive sensors can be formed, for example, by disposing indium tin oxide patterned as electrodes on the substrate. Indium tin oxide is useful for such systems because it is transparent and conductive. Further, it is capable of being deposited in thin layers by way of a printing process. The capacitive sensors may also be deposited on the substrate by electron beam evaporation, physical vapor deposition, or other various sputter deposition techniques.

Another example of a physical sensor is a geo-locator that serves as a location detector 202. In one embodiment, location detector 202 is able to determine location data when an image is captured from a constellation of one or more earth orbiting satellites, or from a network of terrestrial base stations to determine an approximate location. Examples of satellite positioning systems suitable for use with embodiments of the present invention include, among others, the Navigation System with Time and Range (NAVSTAR) Global Positioning Systems (GPS) in the United States of America, the Global Orbiting Navigation System (GLONASS) in Russia, and other similar satellite positioning systems. The satellite positioning systems based location fixes of the location detector 202 autonomously or with assistance from terrestrial base stations, for example those associated with a cellular communication network or other ground based network, or as part of a Differential Global Positioning System (DGPS), as is well known by those having ordinary skill in the art. The location detector 202 may also be able to determine location by locating or triangulating terrestrial base stations of a traditional cellular network, such as a CDMA network or GSM network, or from other local area networks, such as Wi-Fi networks.

Another physical sensor is a heart monitor 203. The heart monitor 203 can be configured to employ EKG or other sensors to monitor a user's heart rate. The heart monitor 203 can include electrodes configured to determine action potentials from the skin of a user.

Another example of a physical sensor is the motion detector 204. Illustrating by example, an accelerometer, gyroscopes, or other device can be used as a motion detector 204 in an electronic device. Using an accelerometer as an example, an accelerometer can be included to detect motion of the electronic device. Additionally, the accelerometer can be used to sense some of the gestures of the user, such as one talking with their hands, running, or walking.

The motion detector 204 can also be used to determine the spatial orientation of an electronic device as well in three-dimensional space by detecting a gravitational direction. In addition to, or instead of, an accelerometer, an electronic compass can be included to detect the spatial orientation of the electronic device relative to the earth's magnetic field. Similarly, one or more gyroscopes can be included to detect rotational motion of the electronic device.

Another example of a physical sensor is a force sensor 205. The force sensor can take various forms. For example, in one embodiment, the force sensor comprises resistive switches or a force switch array configured to detect contact with either the display or the housing of an electronic device. The array of resistive switches can function as a force-sensing layer, in that when contact is made with either the surface of the display or the housing of the electronic device, changes in impedance of any of the switches may be detected. The array of switches may be any of resistance sensing switches, membrane switches, force-sensing switches such as piezoelectric switches, or other equivalent types of technology. In another embodiment, the force sensor can be capacitive. In yet another embodiment, piezoelectric sensors can be configured to sense force as well. For example, where coupled with the lens of the display, the piezoelectric sensors can be configured to detect an amount of displacement of the lens to determine force. The piezoelectric sensors can also be configured to determine force of contact against the housing of the electronic device rather than the display.

Another example of one or more other sensors 113 includes proximity sensors. The proximity sensors fall in to one of two camps: active proximity sensors and “passive” proximity sensors. These are shown as proximity detector components 206 and proximity sensor components 207 in FIG. 2. Either the proximity detector components 206 or the proximity sensor components 207 can be generally used for gesture control and other user interface protocols, some examples of which will be described in more detail below.

As used herein, a “proximity sensor component” comprises a signal receiver only that does not include a corresponding transmitter to emit signals for reflection off an object to the signal receiver. A signal receiver only can be used due to the fact that a user's body or other heat generating object external to device, such as a wearable electronic device worn by user, serves as the transmitter. Illustrating by example, in one the proximity sensor components 207 comprise a signal receiver to receive signals from objects external to the housing of an electronic device. In one embodiment, the signal receiver is an infrared signal receiver to receive an infrared emission from an object such as a human being when the human is proximately located with the electronic device. In one or more embodiments, the proximity sensor component is configured to receive infrared wavelengths of about four to about ten micrometers. This wavelength range is advantageous in one or more embodiments in that it corresponds to the wavelength of heat emitted by the body of a human being.

Additionally, detection of wavelengths in this range is possible from farther distances than, for example, would be the detection of reflected signals from the transmitter of a proximity detector component. In one embodiment, the proximity sensor components 207 have a relatively long detection range so as to detect heat emanating from a person's body when that person is within a predefined thermal reception radius. For example, the proximity sensor component may be able to detect a person's body heat from a distance of about ten feet in one or more embodiments. The ten-foot dimension can be extended as a function of designed optics, sensor active area, gain, lensing gain, and so forth.

Proximity sensor components 207 are sometimes referred to as a “passive IR system” due to the fact that the person is the active transmitter. Accordingly, the proximity sensor component 207 requires no transmitter since objects disposed external to the housing deliver emissions that are received by the infrared receiver. As no transmitter is required, each proximity sensor component 207 can operate at a very low power level. Simulations show that a group of infrared signal receivers can operate with a total current drain of just a few microamps.

In one embodiment, the signal receiver of each proximity sensor component 207 can operate at various sensitivity levels so as to cause the at least one proximity sensor component 207 to be operable to receive the infrared emissions from different distances. For example, the one or more processors (104) can cause each proximity sensor component 207 to operate at a first “effective” sensitivity so as to receive infrared emissions from a first distance. Similarly, the one or more processors (104) can cause each proximity sensor component 207 to operate at a second sensitivity, which is less than the first sensitivity, so as to receive infrared emissions from a second distance, which is less than the first distance. The sensitivity change can be effected by causing the one or more processors (104) to interpret readings from the proximity sensor component 207 differently.

By contrast, proximity detector components 206 include a signal emitter and a corresponding signal receiver. While each proximity detector component 206 can be any one of various types of proximity sensors, such as but not limited to, capacitive, magnetic, inductive, optical/photoelectric, imager, laser, acoustic/sonic, radar-based, Doppler-based, thermal, and radiation-based proximity sensors, in one or more embodiments the proximity detector components 206 comprise infrared transmitters and receivers. The infrared transmitters are configured, in one embodiment, to transmit infrared signals having wavelengths of about 860 nanometers, which is one to two orders of magnitude shorter than the wavelengths received by the proximity sensor components. The proximity detector components can have signal receivers that receive similar wavelengths, i.e., about 860 nanometers.

In one or more embodiments, each proximity detector component 206 can be an infrared proximity sensor set that uses a signal emitter that transmits a beam of infrared light that reflects from a nearby object and is received by a corresponding signal receiver. Proximity detector components 206 can be used, for example, to compute the distance to any nearby object from characteristics associated with the reflected signals. The reflected signals are detected by the corresponding signal receiver, which may be an infrared photodiode used to detect reflected light emitting diode (LED) light, respond to modulated infrared signals, and/or perform triangulation of received infrared signals.

Another example of a physical sensor is a moisture detector 208. A moisture detector 208 can be configured to detect the amount of moisture on or about the display or the housing of the electronic device. The moisture detector 208 can be realized in the form of an impedance sensor that measures impedance between electrodes. As moisture can be due to external conditions, e.g., perspiration, the moisture detector 208 can function in tandem with ISFETS configured to measure pH or amounts of NaOH in the moisture or a galvanic sensor 209 to determine not only the amount of moisture, but whether the moisture is due to perspiration.

An intelligent imager 210 can be configured to capture an image of an object and determine whether the object matches predetermined criteria. For example, the intelligent imager 210 operate as an identification module configured with optical recognition such as include image recognition, character recognition, visual recognition, facial recognition, color recognition, shape recognition and the like. Advantageously, the intelligent imager 210 can be used as a facial recognition device to determine the identity of one or more persons detected about an electronic device. For example, in one embodiment when the one or more proximity sensor components 207 detect a person, the intelligent imager 210 can capture a photograph of that person. The intelligent imager 210 can then compare the image to a reference file stored in memory (105), to confirm beyond a threshold authenticity probability that the person's face sufficiently matches the reference file. Beneficially, optical recognition allows the one or more processors (104) to execute control operations only when one of the persons detected about the electronic device are sufficiently identified as the owner of the electronic device.

A barometer 211 can sense changes in air pressure due to environmental and/or weather changes. In one embodiment, the barometer 211 includes a cantilevered mechanism made from a piezoelectric material and disposed within a chamber. The cantilevered mechanism functions as a pressure sensitive valve, bending as the pressure differential between the chamber and the environment changes. Deflection of the cantilever ceases when the pressure differential between the chamber and the environment is zero. As the cantilevered material is piezoelectric, deflection of the material can be measured with an electrical current.

The medical history of a user, as well as the determinations made by the various one or more other sensors 113 and/or the skin temperature sensor (112) and skin sensor (120), can be stored in a medical profile 212. Periodic updates can be made to the medical profile 212 as well. The medical profile 212 can be a module operable with the one or more processors (104). Such modules can be configured as sets of instructions stored in the memory (105) that are usable by the one or more processors (104) to execute the various wellness-monitoring functions of the device. Alternatively, the modules could be configured in hardware, such as through programmable logic.

A light sensor 213 can detect changes in optical intensity, color, light, or shadow in the environment of an electronic device. This can be used to make inferences about context such as whether a user is exercising outside or indoors. For example, if the light sensor 213 detects low-light conditions in the middle of the day when the location detector 202 indicates that the electronic device is inside, this can confirm that the device is inside. An infrared sensor can be used in conjunction with, or in place of, the light sensor 213. The infrared sensor can be configured to detect thermal emissions from an environment about an electronic device. Where, for example, the infrared sensor detects heat on a warm day, but the light sensor detects low-light conditions, this can indicate that the electronic device is in a room where the air conditioning is not properly set.

A pulse monitor 214 can be configured to monitor the user's pulse. The pulse monitor 214 lends itself to a wristband configuration because the wrist serves as an advantageous location from which to measure a person's pulse. However, the pulse monitor can be used in the headband and garment configurations as well.

The one or more other sensors 113 can also include an audio capture device 215. In one embodiment, the audio capture device 215 includes one or more microphones to receive acoustic input. While the one or more microphones can be used to sense voice input, voice commands, and other audio input, in some embodiments they can be used as environmental sensors to sense environmental sounds such as rain, wind, and so forth.

In one embodiment, the one or more microphones include a single microphone. However, in other embodiments, the one or more microphones can include two or more microphones. Where multiple microphones are included, they can be used for selective beam steering to, for instance, determine from which direction a sound emanated. Illustrating by example, a first microphone can be located on a first side of the electronic device for receiving audio input from a first direction, while a second microphone can be placed on a second side of the electronic device for receiving audio input from a second direction. The one or more processors (104) can then select between the first microphone and the second microphone to beam steer audio reception toward the user. Alternatively, the one or more processors (1034) can process and combine the signals from two or more microphones to perform beam steering.

In one embodiment, the audio capture device 215 comprises an “always ON” audio capture device. As such, the audio capture device 215 is able to capture audio input at any time that an electronic device is operational. As noted above, in one or more embodiments, the one or more processors, which can include a digital signal processor, can identify whether one or more device commands are present in the audio input captured by the audio capture device 215.

One further example of the one or more other sensors 113 is a hygrometer 216. The hygrometer 216 can be used to detect humidity, which can indicate that a user is outdoors or is perspiring. As noted above, the illustrative physical sensors of FIG. 2 are not comprehensive. Numerous others could be added. For example, a wind-speed monitor could be included to detect wind. Accordingly, the one or more other sensors 113 of FIG. 2 are illustrative only, as numerous others will be obvious to those of ordinary skill in the art having the benefit of this disclosure.

Turning now to FIG. 3, as noted above, in one or more embodiments devices configured in accordance with embodiments of the disclosure include a local cooling system 115. FIG. 3 illustrates a first type of local cooling system 115. As will be described in more detail below with reference to FIG. 5, in one or more embodiments the local cooling system 115 is actuated when a user's skin temperature is above a predefined upper skin temperature threshold.

In the illustrative embodiment of FIG. 3, the local cooling system 115 comprises a fluid pump 301 and a radiator 302. The radiator 302 can be configured with small, i.e., two millimeters or smaller in diameter, conduit arranged as an array or grid across one or more surfaces of the device. This radiator 302 can be coupled to the fluid pump 301, which can pump water, Freon, or other coolants through the radiator 302. In one or more embodiments, the fluid pump 301 further includes a refrigeration engine 303 with which the fluid pump 301 can cause the fluid to be cooled prior to delivery to the radiator 302. The fluid pump 301 and/or refrigeration engine 303 can be integrated into the device in one embodiment. Alternatively, fluid pump 301 and/or refrigeration engine 303 can be tethered to a device and stowed in a pocket or clipped to a garment. Other configurations for the local cooling system 115 will be obvious to those of ordinary skill in the art having the benefit of this disclosure.

Turning now to FIG. 4, illustrated therein is another local cooling system 115 configured in accordance with one or more embodiments of the disclosure. In this illustrative embodiment, the local cooling system 115 comprises a shutter 401 and a menthol diffuser 402. The shutter 401, in one or more embodiments, can selectively open and close one or more openings, thereby allowing menthol to be delivered through the openings to the surface of the device or to a user's skin or clothing. In one or more embodiments, the one or more processors (104) selectively open the shutter 401 and cause the menthol diffuser 402 to emit menthol through the openings.

In one or more embodiments, the shutters 401 can also include a radiator system (similar to that described above with reference to FIG. 3) that allows for translation of menthol from the menthol diffuser 402 to the one or more openings. In one or more embodiments, rather than shutters, the tubing has small pores through which menthol can dissipate when the menthol diffuser 402 is active. The menthol diffuser 402 can further include a connector with which the user could add more menthol after depletion.

Turning now to FIG. 5, illustrated therein is one explanatory device 100 in action. As shown, a user 501 is wearing the device 100 as a headband. The user 501 is exercising, which makes her skin temperature rise.

Embodiments of the disclosure contemplate that it is very important to maintain constant body temperature. If the user's body is unable to maintain a relatively constant temperature, the body can start functioning abnormally. As noted, the user 501 in FIG. 5 is exercising. Vigorous exercise for long duration causes increases in heart and breathing rates. In some situations, the body will send more energy to the muscles being exercised. Additionally, more rapid breathing increases the amount of thermal energy within the body. If the body does not have sufficient water to generate sweat, which evaporates, thereby removing heat, the body may keep increasing in temperature. Accordingly, embodiments of the disclosure contemplate that is can be important to track body temperature. Embodiments of the disclosure contemplate that monitoring skin temperature is a very effective way to monitor body temperature.

Advantageously, in one or more embodiments the device 100 includes a skin temperature sensor (112). While the user 501 is exercising, the skin temperature sensor (112) monitors the skin temperature 505 of the user 501 to determine whether it is within a predefined skin temperature range 504. In one or more embodiments, the predefined skin temperature range 504 is defined by a predefined upper skin temperature threshold 507 and a predefined lower sin temperature threshold 506. Illustrating by example, the predefined skin temperature range 504 may be between a predefined upper skin temperature threshold 507 of 100.4 degrees Fahrenheit and a predefined lower skin temperature threshold 506 of 97 degrees Fahrenheit. This predefined skin temperature range 504 is illustrative only. Other predefined skin temperature ranges will be obvious to those of ordinary skill in the art having the benefit of this disclosure.

In one or more embodiments, where the one or more processors (104) of the device 100 determine, from signals received from the skin temperature sensor (112), that the skin temperature 505 is outside the predefined skin temperature range 504, the one or more processors (104) cause the wireless communication circuit (106) to transmit a climate adjustment request 502 to a climate control device 503.

Illustrating by example, presume the user 501 is exercising in a room of her home. The environmental temperature is set by a climate control device 503, which in this embodiment is a HVAC system. In one or more embodiments, when the user's skin temperature 505 exceeds the predefined upper skin temperature threshold 507, the wireless communication circuit (106) of the device 100 sends a message to the HVAC system requesting that the air conditioning be turned ON.

Advantageously, once the skin temperature sensor (112) determines that the skin temperature 505 is above the predefined upper skin temperature threshold 507, the device 100 can send a climate adjustment request 502 to activate the climate control device 503.

In one or more embodiments, the one or more processors (104) can additionally actuate 508 the local cooling system (115) to cool the skin of the user 501. For example, where the local cooling system (115) comprises a fluid pump (301) and a radiator (302), the one or more processors (104) can actuate 508 the local cooling system (115) by causing the fluid pump 301 to circulate fluid through the radiator (302). Additionally, the one or more processors (104) can actuate 508 the local cooling system (115) by causing the refrigeration engine (303) to cool the fluid as well. By contrast, where the local cooling system (115) comprises a shutter (401) and menthol diffuser (402), the one or more processors (104) can actuate the local cooling system (115) by opening the shutter (401) and causing the menthol diffuser (402) to emit menthol. Of course, a combination of these techniques can also be used.

Once the skin temperature 505 falls back within the predefined skin temperature range 504, in one or more embodiments a second climate adjustment request can be delivered to the climate control device 503 to deactivate the same. Additionally, the local cooling system (115) can be turned OFF. The process can repeat should the skin temperature 505 once again move outside the predefined skin temperature range 504.

In one or more embodiments, one or more processors (104) further cause the wireless communication circuit (106) to transmit a temperature event message 509 to a companion electronic device 510. For example, the user 501 may wish to record health information during exercise sessions in the companion electronic device 510, which is a tablet computer in this illustrative embodiment. As such, in one or more embodiments when the skin temperature 505 moves outside the predefined skin temperature range 504, a temperature event message 509 indicating that this occurred is delivered to the companion electronic device 510. Additionally, in some embodiments the companion electronic device 510, rather than the device 100 itself, can transmit the climate adjustment request 502 to the climate control device 503.

Thus, as shown and described in FIG. 5, once the skin temperature sensor (112) detects that skin temperature 505 is rising and passes above the predefined upper skin temperature threshold 507, it sends a signal in the form of the climate adjustment request 502 to activate a cooling system, which can be the local cooling system (115) and/or the climate control device 503.

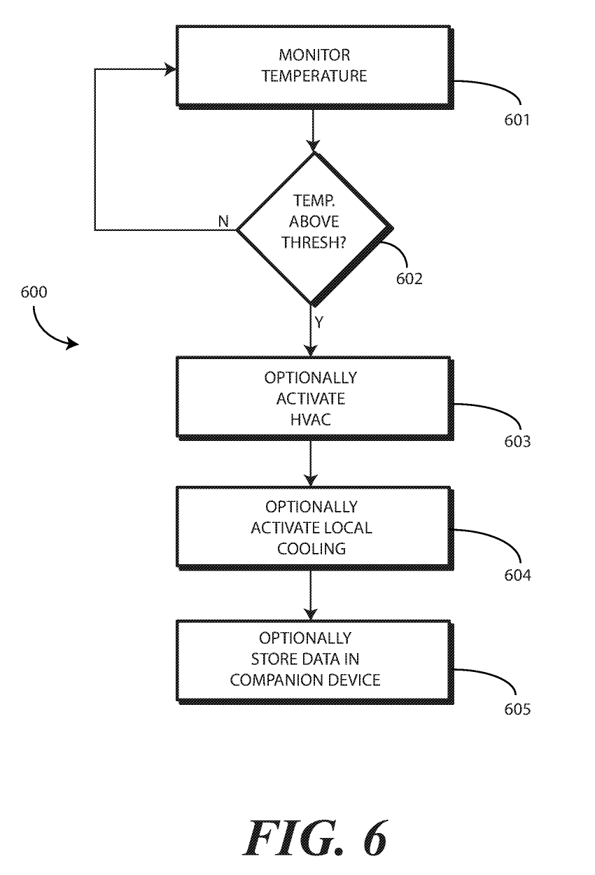

Turning now to FIG. 6, illustrated therein is one explanatory method 600 in accordance with one or more embodiments of the disclosure. Beginning at step 601, the method 600 monitors the temperature of skin using a skin temperature sensor. At decision 602, the method 600 determines or detects whether the skin temperature being monitored at step 601 exceeds a predefined skin temperature threshold. Where it does not, the method 600 returns to step 601 to continue monitoring the skin temperature.

Where the skin temperature is above the predefined skin temperature threshold, which in one or more embodiments is between 100 and 101 degrees Fahrenheit, the method 600 proceeds to step 603. Step 603 includes causing, with one or more processors of a device, a wireless communication circuit to transmit a climate adjustment request to a climate control device. Examples of climate control devices include fans, air conditioners, HVAC systems, and so forth.

In one or more embodiments, the climate adjustment request requests that the climate control device reduce the surrounding temperature to assist in cooling the skin.

In one or more embodiments, optional step 604 comprises actuating a local cooling system to reduce skin temperature. Examples of local cooling systems include elements disposed within the device—rather than those producing airflow or cooler airflow as is the case with the climate control device—that can locally cool skin by being adjacent or in contact with the skin. Thus, a local cooling system can include a menthol emitter, a radiator or other conduit array that circulates fluid, which may be cooled, or other devices.

Accordingly, in one or more embodiments step 604 comprises emitting menthol. Where the emission of menthol is controlled by opening or closing a shutter, step 604 can comprise opening the shutter. In other embodiments, step 604 can comprise circulating water, Freon, or other fluids through a conduit array or radiator. Step 604 can further include cooling the fluid during, or before, circulating it through the conduit array. Other techniques for actuating a local cooling system will be obvious to those of ordinary skill in the art having the benefit of this disclosure.

At optional step 605, the method 600 can comprise causing a wireless communication circuit to transmit a message to a companion electronic device. The message can take various forms. In one embodiment, the message might include a request for a climate control device to reduce the temperature in the surrounding environment. In other embodiments, the message may be a temperature event message or wellness message used to record health information corresponding to the elevated skin temperature in the companion electronic device. Other types of messages will be obvious to those of ordinary skill in the art having the benefit of this disclosure.

The method 600 of FIG. 6 provides steps and decisions for detecting that body temperature, via a measurement of skin temperature, is rising and that the skin temperature is above a predefined skin temperature threshold. When this occurs, the method 600 sends one or more signals or messages to activate a climate cooling system, such as an air conditioner, in one or more embodiments. Alternatively, when this occurs, the method sends one or more signals to a local cooling system. The local cooling system may contain non-active agents and a mixture of polymer and menthol responsible to make skin cooler. Of course, combinations of climate control device cooling and local cooling system cooling can be used to cool the skin. Once body temperature falls below the predefined upper skin temperature threshold, the method 600 then automatically deactivates one or both of the climate control device or the local cooling system. In this way, the method 600 either uses internal cooling mechanism or gets helps from “Internet of Things” devices, such as an air conditioner or fan with wireless communication capabilities, to reduce the body temperature into a desired range.

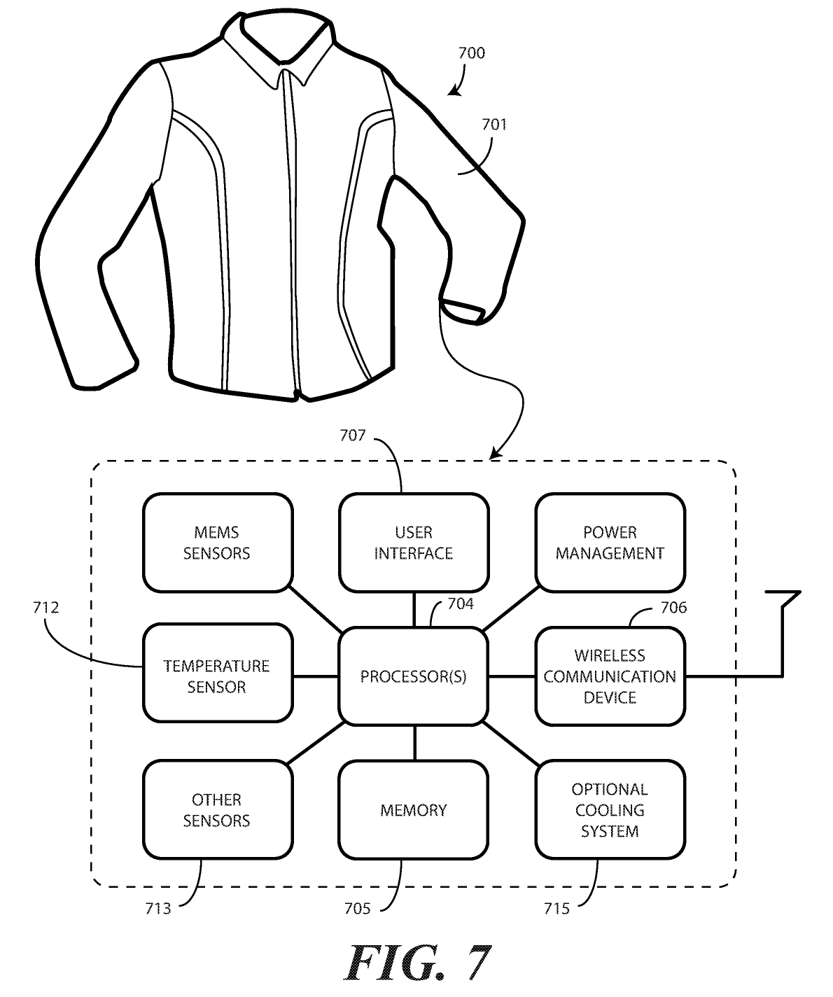

Turning now to FIG. 7, illustrated therein is another device 700 configured in accordance with one or more embodiments of the disclosure. In this illustrative embodiment, the device 700 is configured as a garment 701. For illustration purposes, the garment 701 is shown as a top, such as a shirt or jacket. However, it should be understood that the garment 701 can be configured in other ways, such as a shirt, undergarment, pair of pants, pair of shorts, sweater, pullover, and so forth. Still other garment configurations will be obvious to those of ordinary skill in the art having the benefit of this disclosure.

The various components of the device 700 can be integrated into the garment 701 or disposed in pockets or other holding locations. For example, in one or more embodiments the skin temperature sensor 712 can be distributed across surfaces of the sleeves and body of the garment so as to sense skin temperature at different locations. Physical connections or wireless connections can then communicate skin temperature signals back to the one or more processors 704, which may be disposed in a pocket or other compartment.

As with the device (100) of FIG. 1, the garment 701 of FIG. 7 includes one or more processors 704. The one or more processors 704 are operable with other components of the garment 701. The one or more processors 704 can include a microprocessor, a group of processing components, one or more ASICs, programmable logic, or other type of processing device. The one or more processors 704 can be configured to process and execute executable software code to perform the various functions of the device 700.

A storage device, such as memory 705, can optionally store the executable software code used by the one or more processors 704 during operation. The memory 705 may include either or both static and dynamic memory components, may be used for storing both embedded code and user data. The software code can embody program instructions and methods to operate the various functions of the garment 701, and also to execute software or firmware applications and modules. The one or more processors 704 can execute this software or firmware, and/or interact with modules, to provide device functionality.

The garment 701 can have a user interface 707. For example, the garment 701 can include a plurality of visual indicators disposed thereon to show the status of the circuit components. In one or more embodiments, the one or more processors 704 may use proximity sensors or other sensors 713 (such as those described above with reference to FIG. 2) to detect gesture input or touch input. Feedback can be delivered to the user by delivering control signals to illuminate one or more of the visual indicators with a duration, intensity, color, direction, or other characteristic to provide user input. A flexible display can also be mounted on the garment 701 as well.

The garment 701 further includes a wireless communication circuit 706 that can be configured for wired or wireless communication with one or more other devices or networks. The networks can include a wide area network, a local area network, and/or personal area network as previously described. The wireless communication circuit 706 can include wireless communication circuitry, one of a receiver, a transmitter, or transceiver, and one or more antennas.

Other components can be included with the garment 701. The other components can include input and output components associated with a user interface 707, such as power inputs and outputs, audio inputs and outputs, and/or mechanical inputs and outputs. The other components can include output components such as video, audio, and/or mechanical outputs. Other examples of output components include audio output components such as a loudspeaker disposed behind a speaker port or other alarms and/or buzzers and/or a mechanical output component such as vibrating or motion-based mechanisms. Still other components will be obvious to those of ordinary skill in the art having the benefit of this disclosure.

One or more sensor circuits are operable with the one or more processors 704 in one or more embodiments. In one or more embodiments, the sensor circuits comprise one or more skin temperature sensors 712 that detect the amount of thermal energy being generated by the skin of a person wearing the garment 701. Examples of skin temperature sensors 712 include thermistors, thermocouples, thermometers, resistive temperature detectors, silicon band gap temperature sensors, integrated circuit temperature sensors, bimetallic thermostats, infrared signal sensors, and so forth. As noted above, they can be distributed across the inner surfaces of the garment 701 in one or more embodiments so that they can directly contact the skin of the user. The skin temperature sensors 712 can be operable with a skin sensor as previously described.

One or more other sensors 713 can be included with the garment 701. The one or more other sensors 713 can also be configured to sense or determine physical parameters indicative of conditions in an environment about the garment 701. Examples of such other sensors 713 were described above with reference to FIG. 2 and will not be repeated here in the interest of brevity.

In one or more embodiments, a local cooling system 715 can be incorporated into the garment 701. The one or more processors 704 actuate the local cooling system 715 when the skin temperature is above a predefined upper skin temperature threshold.

The local cooling system 715 can include a network of flexible conduits that circulate or emit liquid to cool the skin. For example, the local cooling system 715 can include a pump to emit a small amount of menthol out the garment's network of flexible conduit or tubing. The one or more processors 704 can include a wireless system as well where it can read other body-worn body temperature sensors, such as one disposed within a smart watch, and factor it into the users overall body temperature when controlling the local cooling system 715. In one or more embodiments, the garment 701 includes the local cooling system 715 by being made special fiber optics which trigger waves to cool down the body temperature. Where the local cooling system 715 includes a pump, the pump can be integrated into the garment 701 or alternatively tethered to the garment 701 where is can be clipped to the garment 701 or placed into a pocket. The local cooling system 715 can also include a processor to accept commands such as activate or deactivate.

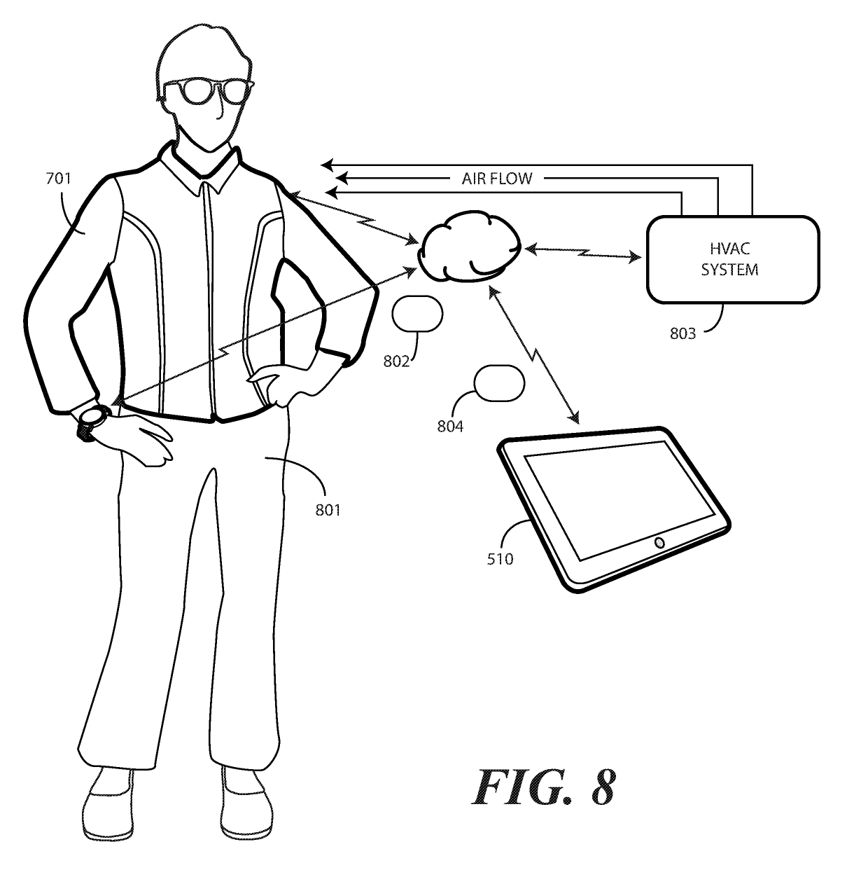

Turning now to FIG. 8, illustrated therein is the garment 701 in use. In this embodiment, as shown, a user 801 is wearing the garment 701. The room in which the user 801 is standing is relatively cold, which reduces her skin temperature.

As before, the skin temperature sensor (712) monitors the skin temperature of the user 801 to determine whether it is within a predefined skin temperature range (504). The predefined skin temperature range (504) is defined by a predefined upper skin temperature threshold (507) and a predefined lower sin temperature threshold (506). Where the one or more processors (704) of the garment 701 determine, from signals received from the skin temperature sensor (712), that the skin temperature (505) is outside the predefined skin temperature range (504), the one or more processors (704) cause the wireless communication circuit (706) to transmit a climate adjustment request 802 to a climate control device 803.

In this illustrative embodiment, the user 801 is cold. The environmental temperature is set by the climate control device 803, which in this embodiment is a HVAC system. In one or more embodiments, when the user's skin temperature falls below the predefined lower skin temperature threshold (506), the wireless communication circuit (706) of the garment 701 sends a message to the HVAC system requesting that either the air conditioning be turned OFF or the heat be turned ON. Advantageously, once the skin temperature sensor (712) determines that the skin temperature is below the predefined lower skin temperature threshold (506), the garment 701 can send a climate adjustment request 802 to activate warming elements of the climate control device 803.

If the reverse case were true, i.e., if the user 801 was hot, with a skin temperature above the predefined upper skin temperature threshold (507), the garment 701 could send a climate adjustment request 802 to activate cooling elements of the climate control device 803. Additionally, the one or more processors (704) could actuate the local cooling system (715) to cool the skin of the user 501. For example, where the local cooling system (715) comprises a fluid pump and a radiator, the one or more processors (704) can actuate the local cooling system (715) by causing the fluid pump to circulate fluid through the radiator. Additionally, the one or more processors (704) can actuate the local cooling system (715) by causing a refrigeration engine to cool the fluid as well. Where the local cooling system (715) comprises a shutter and menthol diffuser, the one or more processors (704) can actuate the local cooling system (715) by opening the shutter and causing the menthol diffuser to emit menthol. Of course, a combination of these techniques can also be used.

Regardless of whether a warming or cooling operation is performed, once the skin temperature falls back within the predefined skin temperature range (504), in one or more embodiments a second climate adjustment request can be sent to terminate the warming or cooling operation. Where a cooling operation is performed with the local cooling system (715), the local cooling system (715) can be turned OFF. The process can repeat should the skin temperature once again move outside the predefined skin temperature range (504).

In one or more embodiments, one or more processors (704) further cause the wireless communication circuit (706) to transmit a temperature event message 804 to a companion electronic device 510. For example, the user 801 may wish to record health information during exercise sessions in the companion electronic device 510. As such, in one or more embodiments when the skin temperature moves outside the predefined skin temperature range (504), a temperature event message 804 indicating that this occurred is delivered to the companion electronic device 510. Additionally, in some embodiments the companion electronic device 510, rather than the garment 701, can transmit the climate adjustment request 802 to the climate control device 803.

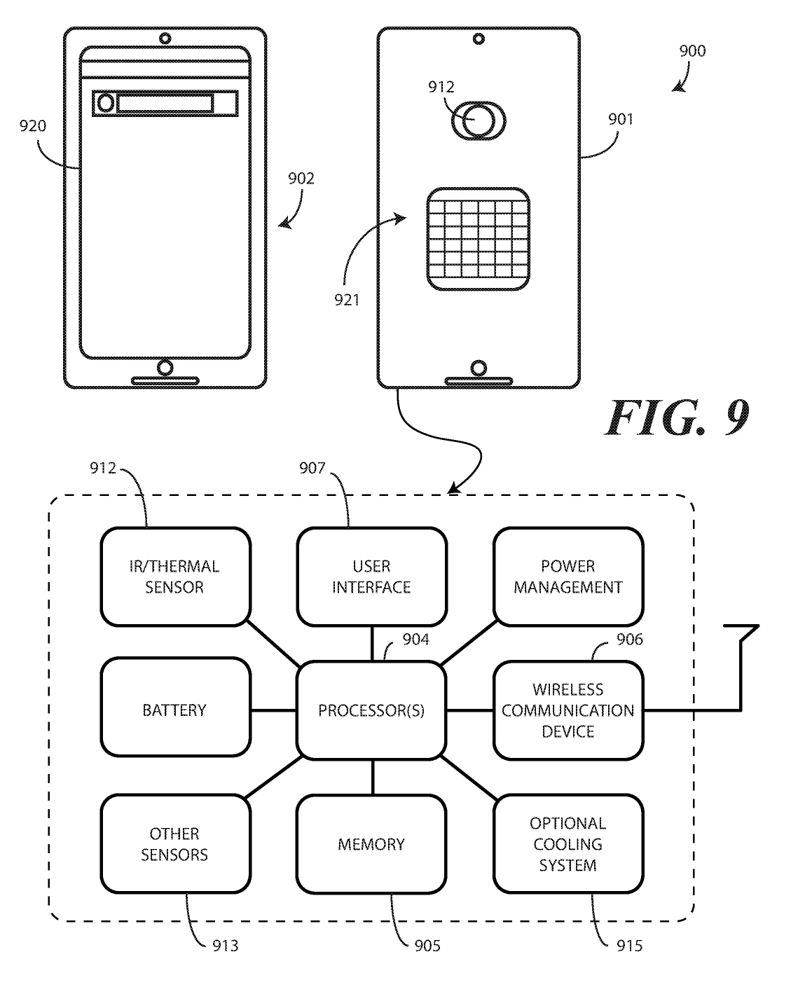

Turning now to FIG. 9, illustrated therein is another device 900 configured to monitor skin temperature of a user and perform cooling or warming operations. In this illustrative embodiment, the device 900 is configured as an attachment 901 that works in conjunction with an electronic device 902, which is shown illustratively as a smartphone. The attachment 901 is selectively attachable and detachable from the electronic device 902. When attached, the attachment 901 brings increased functionality to the electronic device 902 beyond its inherent capabilities. While the electronic device 902 is shown as a smartphone, it take other forms as well, including as a palm top computer, a gaming device, a laptop computer, a multimedia player, and so forth. Still other examples of electronic devices will be obvious to those of ordinary skill in the art having the benefit of this disclosure.

As with the device (100) of FIG. 1 and the device (700) of FIG. 7, the attachment 901 of

FIG. 9 includes one or more processors 904. The one or more processors 904 are operable with other components of the attachment 901. The one or more processors 904 can include a microprocessor, a group of processing components, one or more ASICs, programmable logic, or other type of processing device. The one or more processors 904 can be configured to process and execute executable software code to perform the various functions of the attachment 901.

A storage device, such as memory 905, can optionally store the executable software code used by the one or more processors 904 during operation. The memory 905 may include either or both static and dynamic memory components, may be used for storing both embedded code and user data. The software code can embody program instructions and methods to operate the various functions of the attachment 901, and also to execute software or firmware applications and modules. The one or more processors 904 can execute this software or firmware, and/or interact with modules, to provide device functionality.

The attachment 901 can have a user interface 907. Alternatively, since the attachment 901 is coupled to an electronic device 902 when in use, the display 920 of the electronic device 902 can be used as the user interface 907 of the attachment 901. Similarly, the attachment 901 can include a wireless communication circuit 906 that can be configured for wired or wireless communication with one or more other devices or networks. The networks can include a wide area network, a local area network, and/or personal area network as previously described. Alternatively, the attachment 901 can use wireless communication circuitry of the electronic device 902 for communication with other devices.

Other components can be included with the attachment 901. The other components can include input and output components associated with a user interface 907, such as power inputs and outputs, audio inputs and outputs, and/or mechanical inputs and outputs. The other components can include output components such as video, audio, and/or mechanical outputs. Other examples of output components include audio output components such as a loudspeaker disposed behind a speaker port or other alarms and/or buzzers and/or a mechanical output component such as vibrating or motion-based mechanisms. Still other components will be obvious to those of ordinary skill in the art having the benefit of this disclosure.

One or more sensor circuits are operable with the one or more processors 904 in one or more embodiments. In one or more embodiments, the sensor circuits comprise one or more skin temperature sensors 912 that detect the amount of thermal energy being generated by the skin of a person wearing near the attachment 901. In this illustrative embodiment, since the attachment 901 is generally not in contact with the skin, the one or more skin temperature sensors 912 comprise infrared signal sensors that can determine temperature without contacting the skin.

One or more other sensors 913 can be included with the attachment 901. The one or more other sensors 913 can also be configured to sense or determine physical parameters indicative of conditions in an environment about the attachment 901. Examples of such other sensors 913 were described above with reference to FIG. 2 and will not be repeated here.

In one or more embodiments, a local cooling system 915 can be incorporated into the attachment 901. The one or more processors 904 actuate the local cooling system 915 when the skin temperature is above a predefined upper skin temperature threshold.

Since the attachment 901 is generally not in contact with the skin, where the local cooling system 915 is included, it can be configured to circulate air. In this illustrative embodiment, the local cooling system 915 comprises a fan 921, carried by the housing of the attachment 901. In this illustrative embodiment, to prevent user contact with the fan 921, the fan 921 is disposed behind a grille. The housing of the attachment 901 may include one or more ducts through which the fan 921 may draw or push air.

In one or more embodiments, the attachment 901 can be selectively attached to, or detached from, the electronic device 902. In one or more embodiments, the housing of the attachment 901 is selectively attachable to the electronic device 902 by one or more coupling devices. In one or more embodiments, the housing of the attachment 901 can be mechanically attached to the electronic device 902. For example, mechanical clasps for the attachment 901 can be configured to wrap about, or engage, the housing of the electronic device 902, thereby retaining the attachment 901 against a surface of the electronic device 902. Such clasps permit the attachment 901 to be completely detached from the electronic device 902 and treated as an accessory.

In another embodiment, when not in use, the attachment 901 may be mechanically retained to the electronic device 902 by a lanyard or similar device. Such a configuration helps to prevent inadvertent loss of the attachment 901 when detached from the housing of the electronic device 902.

In yet another embodiment, the attachment 901 may be coupled to the electronic device 902 by a hook and slider mechanism so as to be detachable from the housing yet non-detachable from the electronic device 902 itself. Other attachment mechanisms include magnetic couplings, snaps, protective casing couplings, boot couplings, static attachment connectors, vertical locators, horizontal locators, and the like. Some of these various mechanical configurations will be illustrated in more detail below. These mechanical embodiments are intended to be illustrative only. As an alternate to mechanical attachments, the attachment 901 can be attached to the electronic device 902 using static adhesion, mechanical suction, or in other ways.

In one or more embodiments, the electronic device 902 and the attachment 901 can even include complementary or common components. For example, the electronic device 902 and attachment 901 may both include components for receiving user input, such as loudspeakers, microphones, earpiece speakers, and the like. An electrical connection can exist between the electronic device 902 and the attachment 901 so that communication can occur between the attachment 901 and the electronic device 902.

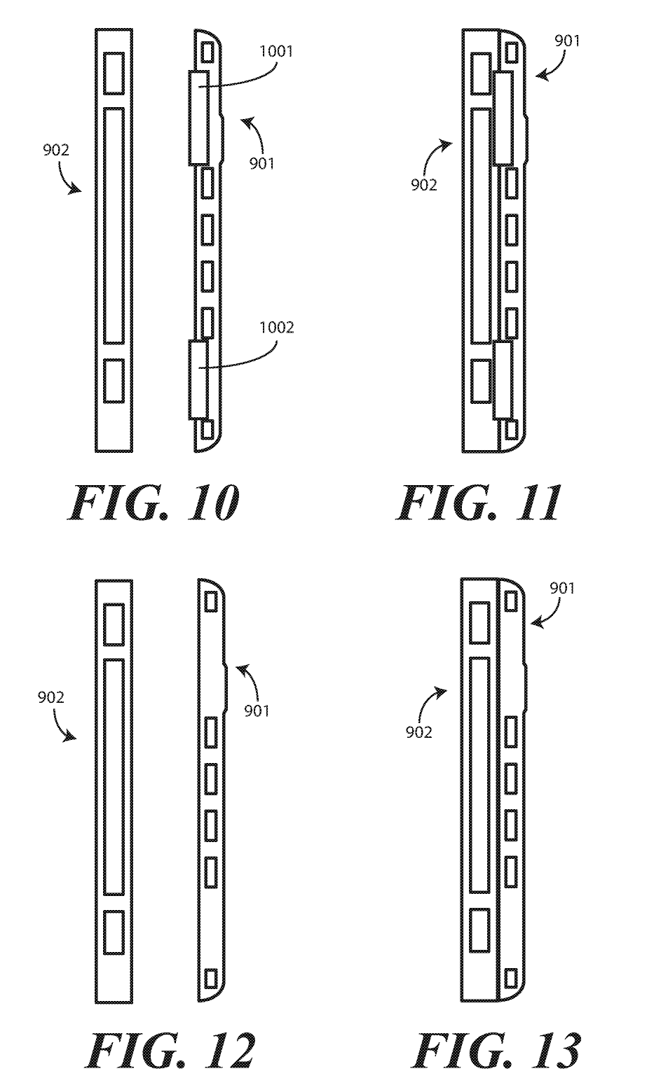

Turning now to FIGS. 10-13, illustrated therein are examples of various ways in which an attachment can be coupled to an electronic device in accordance with one or more embodiments of the disclosure. As noted above, in one or more embodiments of the disclosure, the attachment can be coupled to the electronic device by mechanical, magnetic, suction, static, and other techniques.

Beginning with FIG. 10, in one or more embodiments, the housing of the attachment 901 can be mechanically attached to the electronic device 902 by one or more coupling devices. In this illustrative embodiment, the coupling devices comprise mechanical clasps 1001,1002 that are configured to wrap about, or engage, the housing of the electronic device 902, thereby retaining the attachment 901 against the major surface defined by the back side of the electronic device 902. Such mechanical clasps 1001,1002 permit the attachment 901 to be completely detached from the electronic device 902 and treated as a separate accessory. In FIG. 10, the attachment 901 is shown detached from the electronic device 902, while in FIG. 11 the attachment 901 is shown attached to the electronic device 902 to form a modular system.

Turning now to FIG. 12, another coupling system is shown. In the illustrative embodiment of FIG. 12, the backside of the electronic device 902 includes one or more alignment features configured and placed to mate with complementary mating features on the front side of the attachment 901. In one or more embodiments, the alignment features and complementary mating features are magnetic such that the front side of the attachment 901 can be magnetically adhered to the backside of the electronic device 902. In FIG. 12, the attachment 901 is shown detached from the electronic device 902, while in FIG. 13 the attachment 901 is shown attached to the electronic device 902 to form a modular system.

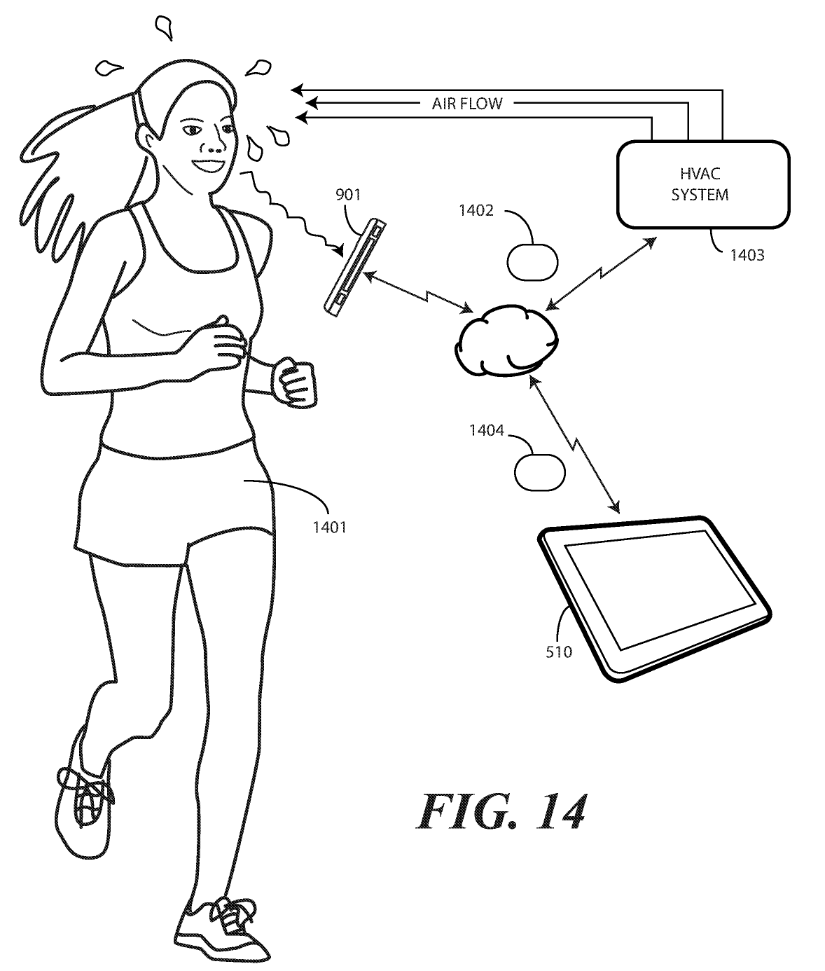

Turning now to FIG. 14, illustrated therein is the attachment 901 in use. In this illustrative embodiment, the attachment 901 does not contact the skin of the user 1401. Instead, it is placed within a thermal reception radius within which the skin temperature sensor (912) can receive thermal signals to detect the skin temperature of the user 1401. For example, since the user 1401 is running, the attachment 901 may be placed on the handles of a treadmill.

As previously described, when the one or more processors (904) determine, with signals received from the skin temperature sensor (912), a skin temperature that is above a predefined upper skin temperature threshold, they cause the wireless communication circuit (906) to transmit a cooling request 1402 to a climate control device 1403. The climate control device 1403 in this embodiment is either a fan or an air conditioner. Moreover, where a local cooling system (915) is included, the one or more processors can actuate the local cooling system (915) to reduce the skin temperature as well. Additionally, the one or more processors (904) can further cause the wireless communication circuit (906) to deliver a skin temperature elevation occurrence 1404 across a network to a companion electronic device 510.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims. Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

What is claimed is:

1. A device, comprising:
a skin temperature sensor;
one or more processors operable with the skin temperature sensor; and
a wireless communication circuit operable with the one or more processors;
the one or more processors determining, with signals received from the skin temperature sensor, a skin temperature that is outside a predefined skin temperature range, and where the skin temperature is outside the predefined skin temperature range, causing the wireless communication circuit to transmit a climate adjustment request to a climate control device.

2. The device of claim 1, the predefined skin temperature range defined by a predefined upper skin temperature threshold and a predefined lower skin temperature threshold.

3. The device of claim 2, further comprising a local cooling system operable with the one or more processors, the one or more processors actuating the local cooling system when the skin temperature is above the predefined upper skin temperature threshold.

4. The device of claim 3, the local cooling system comprising a shutter and a menthol diffuser.

5. The device of claim 4, the one or more processors actuating the local cooling system by opening the shutter and causing the menthol diffuser to emit menthol.

6. The device of claim 3, the local cooling system comprising a fluid pump and radiator.

7. The device of claim 6, the one or more processors actuating the local cooling system by causing the fluid pump to circulate a fluid through the radiator.

8. The device of claim 7, the one or more processors further causing the fluid pump to cool the fluid.

9. The device of claim 3, wherein the device is configured as a headband.

10. The device of claim 3, wherein the device is configured as a garment.

11. The device of claim 3, wherein the device is configured as an attachment for an electronic device, further wherein the skin temperature sensor comprises an infrared temperature sensor.

12. The device of claim 3, the one or more processors further causing the wireless communication circuit to transmit a temperature event message to a companion electronic device.

13. A method, comprising:
detecting, with a skin temperature sensor, a skin temperature being above a predefined skin temperature threshold;
causing, with one or more processors, when the skin temperature is above the predefined skin temperature threshold, a wireless communication circuit to transmit a climate adjustment request to a climate control device; and
actuating, with the one or more processors, a local cooling system to reduce the skin temperature.

14. The method of claim 13, wherein the actuating comprises emitting menthol.

15. The method of claim 14, wherein the actuating further comprises opening a shutter.

16. The method of claim 13, wherein the actuating comprises circulating water through a conduit array.

17. The method of claim 16, wherein the actuating further comprises cooling the water.

18. A device, comprising:
a skin temperature sensor;
one or more processors operable with the skin temperature sensor;
a wireless communication circuit operable with the one or more processors; and
a local cooling system operable with the one or more processors;
the one or more processors:
determining, with signals received from the skin temperature sensor, a skin temperature that is above a predefined skin temperature threshold, and where the skin temperature is above the predefined skin temperature threshold, causing the wireless communication circuit to transmit a cooling request request to a climate control device; and
actuating the local cooling system to reduce the skin temperature.

19. The device of claim 18, wherein the climate control device comprises one of a fan or an air conditioner.

20. The device of claim 19, the one or more processors further causing the wireless communication circuit to deliver a skin temperature elevation occurrence across a network to a companion electronic device.
